(19) United States
(12) Patent Application Publication
(10) Pub. No.: US 2006/0226112 A1
(43) Pub. Date: Oct. 12, 2006

(54) Liquid vial closure with improved anti-evaporation features

(76) Inventors: Douglas Paul Clark, Newark, DE (US); Jay Mark Walters, New London, PA (US)

Correspondence Address: DADE BEHRING INC.;LEGAL DEPARTMENT, 1717 DEERFIELD ROAD, DEERFIELD, IL, 60015, US

(21) Appl. No.: 11/100,120

(22) Filed: Apr. 6, 2005

(51) Int. Cl. B65D 51/00; A61B 19/00

(52) U.S. Cl. 215/247; 604/415

(57) ABSTRACT

A re-sealing puncturable closure for use with vials having calibration or control solutions chemicals therein adapted for use in a manner that advantageously minimizes evaporation from a solution vial prior to and subsequent to aspiration of solution therefrom.

### FIELD OF THE INVENTION

The present invention relates to processing a patient's biological fluids such as urine, blood serum, plasma, cerebrospinal fluid and the like. More particularly, the present invention relates to a closure for calibration or control, solution vials involved in performing quality control procedures within an automated biochemical analyzer adapted for analyzing biological fluids.

### BACKGROUND OF THE INVENTION

Biochemical analyzers are well known and almost universally employ some sort of a calibration curve that relates analyte concentration within a carefully prepared solution having a known analyte concentration against the signal generated by the reaction monitoring means in response to the presence of the analyte. Such solutions are called “calibrators” or “calibration solutions” or “standard solutions” and are contained in vial-like containers closed with a stopper or closure of some sort. It is regular practice within the biochemical analytical industry to establish a full calibration curve for a chemical analyzer by using multiple calibration solutions or calibrators which have been carefully prepared with known, predetermined concentrations of analyte. These calibration or standard solutions are assayed one or more times and the mean resulting reaction signals are plotted versus their respective known analyte concentrations. A continuous calibration curve is then produced using any of several mathematical techniques chosen to produce an accurate replication of the relationship between a reaction signal and the analyte concentration. The shape of the calibration curve is affected by a complex interaction between reagents, analyte and the analyzer's electromechanical design. Thus, even if the theoretical analyte-reagent reaction is known, it is generally necessary to employ mathematical techniques to obtain an acceptable calibration curve. The range of analyte concentrations used in establishing a full calibration curve is typically chosen to extend below and beyond the range of analyte concentrations expected to be found within biological samples like blood, serum, plasma, urine and the like.

Problematically, certain calibration solutions employed in the industry have an undesirably short useful life time during which the solution remains stable after the vial-container is opened due to evaporation. One solution to this problem is to originally produce the calibration vial with an evaporation-proof closure, usually made of a hard plastic material. When the vial is to be employed in an analyzer, the evaporation-proof closure is replaced with a threaded cap having an open hole in the center portion and a relatively soft rubber-like stopper filling the hole. The rubber stopper is frequently pre-cut with an X-shaped slit opening so as to allow air to enter the vial when the stopper is penetrated by a probe during aspiration. The necessity for allowing air to freely enter the vial during aspiration comes from the adverse effects on the volume of fluid extracted because of a partial vacuum being otherwise formed within the vial when liquid is aspirated. When it is desired to remove a portion of the solution from the vial for calibration or quality control procedures, the solution is aspirated through a probe penetrating through the X-shaped slit in the stopper. Unfortunately, however, it has been found that after aspiration is completed and the probe removed from the stopper, an X-shaped slit opening does not have a desired level of anti-evaporation properties, possibly because the soft rubber does not have a sufficient elasticity modulus to adequately re-close the stopper.

### SUMMARY OF THE INVENTION

The invention provides a re-sealing pre-slit closure for use with vials having calibration or control liquid solutions therein. The stopper-closure of the present invention is adapted for use in a manner that: (1) provides long term anti-evaporation properties for unopened vials; and (2) provides increased anti-evaporation properties for opened vials. The closure has a thin top layer of metallic foil adhered onto a bottom layer of elastomeric material, the bottom layer being pre-slit in a straight line. The layer of metallic foil provides long-term anti-evaporation properties for unopened vials and can easily be punctured by an aspiration probe when it is desired to removed solution from the vial. The layer of elastomeric material has a thickness and a stiffness selected so that: (1) when the aspiration probe is inserted through the line-slit therein, a small air gap is formed around the probe allowing air to enter into the vial as the solution is aspirated therefrom, and (2) the layer of elastomeric material has a thickness and a stiffness selected so that when the probe is removed from the vial after aspiration is completed, the elastomeric material springs back to its original conformation and closes the line-slit therein. It has been found that this combination of features produces a surprising improvement in the on-board use-life of a calibration or control solution by significantly reducing evaporation from an opened vial.

### BRIEF DESCRIPTION OF THE DRAWINGS

The invention will be more fully understood from the following detailed description thereof taken in connection with the accompanying drawings which form a part of this application and in which:

FIG. 1 is a schematic plan view of an automated analyzer adapted to perform the present invention;

FIG. 2 is an enlarged schematic plan view of a portion of the analyzer of FIG. 1;

FIGS. 3A and 3B are perspective views of a calibration and control solution vial rack useful in the analyzer of FIG. 1 illustrating the closure of the present invention;

FIG. 4 is a perspective view of the stopper element of the closure of the present invention;

FIG. 5 is an elevation view of the stopper of FIG. 4;

FIG. 5A is a section view of the stopper of FIG. 5 taken along the line A-A;

FIG. 6 is a section view of the stopper of FIG. 5 taken along the line A-A as employed with a cap of the closure of the present invention;

FIG. 6A is a section view of the stopper of FIG. 5 taken along the line A-A as employed with a cap of the closure of the present invention and penetrated by a probe;

FIG. 7 is a top view of the closure of FIG. 4 illustrating a probe opening the stopper; and,

FIG. 8 is a top view of the closure of FIG. 4 illustrating the probe removed from the stopper after aspiration is completed.

### DETAILED DESCRIPTION OF THE INVENTION

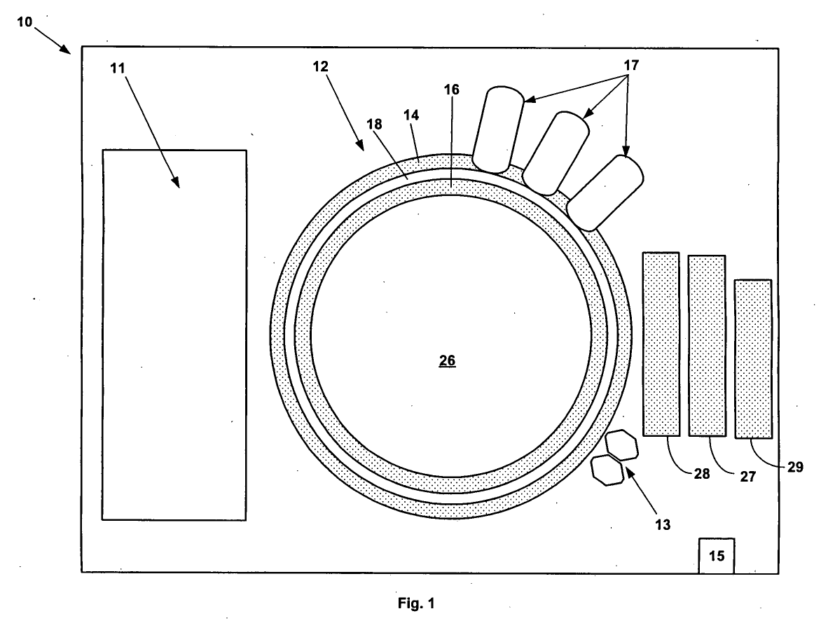

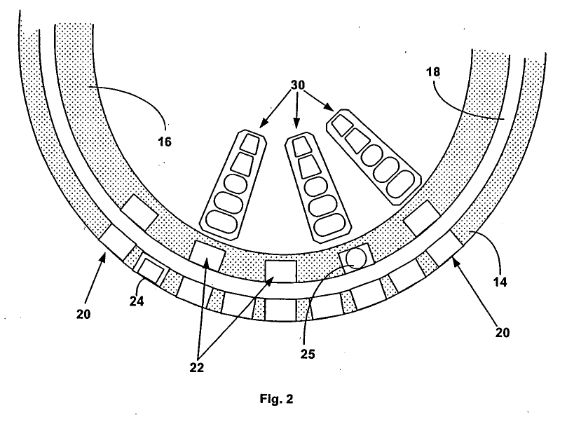

FIG. 1, taken with FIG. 2, shows schematically the elements of an automatic chemical analyzer 10 in which the present invention may be advantageously practiced, analyzer 10 comprising a reaction carousel 12 supporting an outer cuvette carousel 14 having cuvette ports 20 formed therein and an inner cuvette carousel 16 having vessel ports 22 formed therein, the outer cuvette carousel 14 and inner cuvette carousel 16 being separated by a open groove 18. Sample transport and aliquot zone 11 Cuvette ports 20 are adapted to receive a plurality of reaction cuvettes 24 that contain various reagents and sample liquids for conventional clinical and immunoassay assays while vessel ports 22 are adapted to receive a plurality of reaction vessels 25 that contain specialized reagents for ultra-high sensitivity luminescent immunoassays. Reaction carousel 12 is rotatable using stepwise movements in a constant direction, the stepwise movements being separated by a constant dwell time during which carousel 12 is maintained stationary and computer controlled assay operational devices 13, such as sensors, reagent add stations, mixing stations and the like, operate as needed on an assay mixture contained within a cuvette.

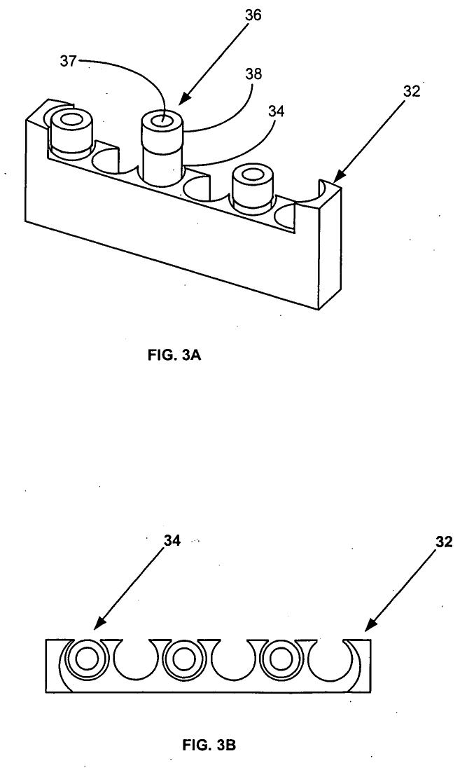

Temperature-controlled storage areas or servers 26, 27 and 28 store a plurality of multi-compartment elongate reagent cartridges 30 like that described in co-pending application Ser. No.: 09/949,132 assigned to the assignee of the present invention, cartridges 30 containing reagents as necessary to perform a given assay. Server 26, also stores calibration and quality control solution vial racks 32 like seen in FIGS. 3A and 3B having calibration or quality control solutions in vials 34 to be used in calibration and quality control procedures by analyzer 10.

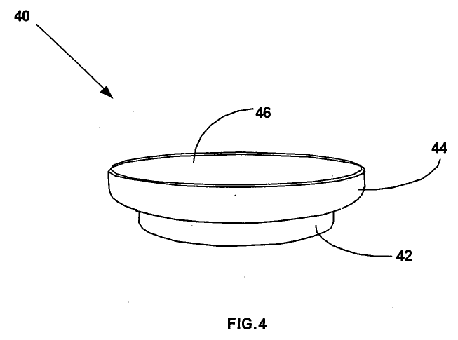

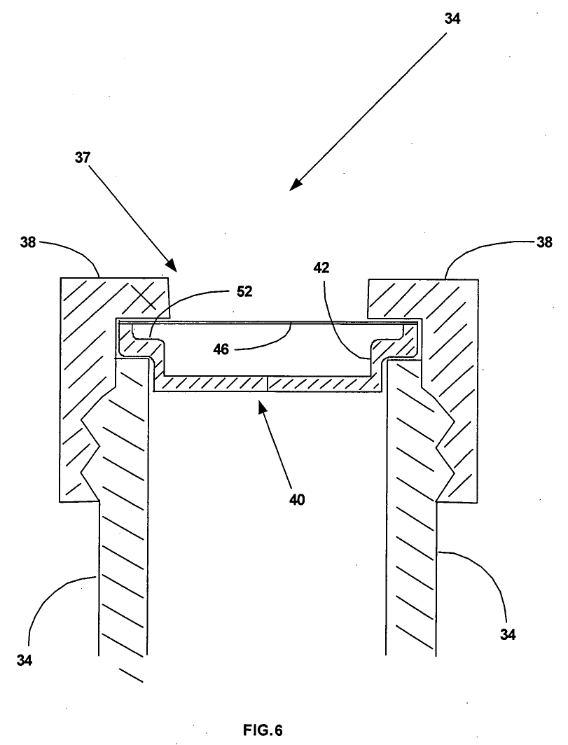

It is known in the industry that the so-called shelf-life of certain calibration and control chemical solutions, shelf-life being the length of time a chemical solution may be stored in a controlled environment and retain its chemical properties within its specified useful range, is too short for the solution to be stored in open or partially closed vial-like containers on-board analyzer 10. The present invention extends the shelf-life of certain calibration and control chemical solutions by providing a re-sealing, easily puncturable closure 36 for calibration vial-like containers 34, closure 36 comprising a cap element 38 sized to fit over and retain a stopper element 40, cap 38 being threaded and typically formed of hard polymer and heteropolymer resins and having an opening 37 to permit a probe to pass therethrough and penetrate stopper 40 (FIGS. 3A, 4 and 6). Cap element 38 may be formed from a number of different resins, including polyolefins, low density polyethylene, high impact polystyrene and polycarbonate. Cap element 38 can also be comprised of a combination of such resins. FIG. 4 is a perspective view of the puncturable, resealable stopper 40 element of the present invention, stopper 40 having a lowermost circular trunk portion 42 sized to fit into and seal the top opening of calibration vial 34, like seen in FIG. 6. Trunk portion 42 depends from an open-bottom, outermost circular band portion 44 of stopper 40, circular band portion 44 having an open top sealed with a metallic foil 46.

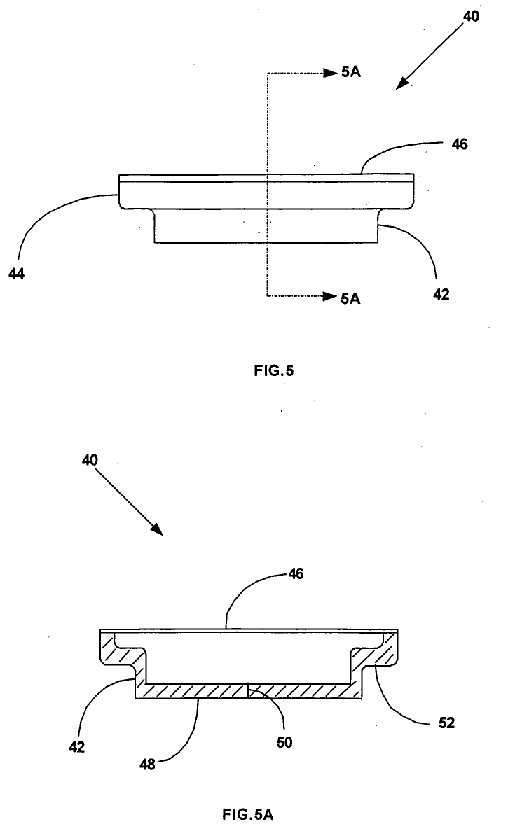

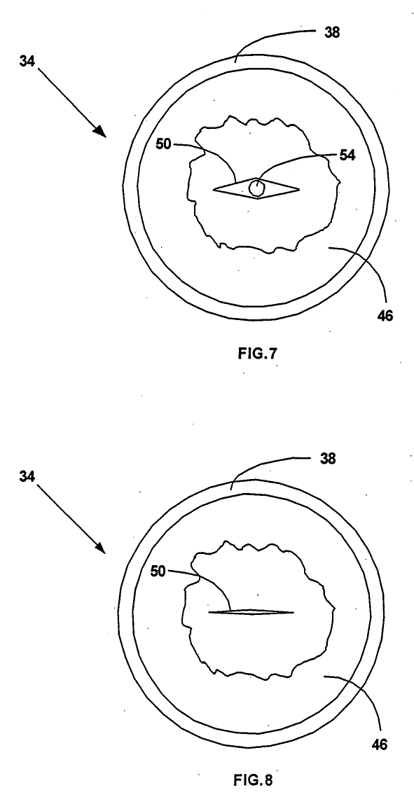

FIG. 5 is an elevation view of stopper 40 further illustrating these features and FIG. 5A is a section view along the line A-A in FIG. 5 further illustrating an important feature of stopper 40 in providing a re-sealing function of stopper 40. Trunk portion 42 is seen to have a closed bottom portion 48 with a slit 50 cut therethrough to facilitate penetration of the closed bottom portion 48 of stopper 40 by an aspiration probe (FIG. 7). The lengthwise dimension of slit 50 may be seen in FIG. 8. As seen in FIG. 6, trunk portion 42 further has an outwardly extending upper shoulder portion 52 sized to prevent trunk 42 from being pushed entirely into vial 30V. Stopper element 40 of the present invention is thus seen to comprise a lowermost circular trunk portion 42 joined to an outwardly extending upper shoulder portion 52, the shoulder portion 52 depending from an outermost circular band portion 44, the circular band portion 44 having an open top sealed with a non-air permeable metallic foil 46.

FIG. 6 further shows closure 36 threaded over the open top of vial 34 illustrating how the shoulder portion 52 acts as a seal between cap 38 and vial 34 when closure 34 of the present invention is used to close vial 34 prior to aspiration of any calibration or control solution therefrom. Because metallic foil 46 is unbroken, solution within vial 34 is not exposed to air and closure 34 is effective in preventing evaporation of solution therefrom. Note that the foil 46 is protected from accidental tearing because it is recessed below the upper surface of cap 38. FIG. 6A illustrates a probe 54 having easily torn aside foil 46 and penetrated slit 50 of stopper 40 during aspiration of calibration or control solution from vial 34. FIG. 7 is a top view of the probe 54 having penetrated slit 50 and illustrated an important feature of the present invention as an air gap 56 formed between probe 54 and stopper 40, the air gap effective to allow air into the interior of vial 34 during aspiration of solution so that air pressure is equalized on both sides of stopper 40, eliminating an vacuum buildup within vial 34 and adversely affecting the amount of aspirated solution.

FIG. 8 illustrates an advantage of the closure 34 of the present invention after probe 54 is withdrawn from slit 50 of stopper 40 after aspiration of calibration or control solution from vial 34, wherein slit 50 is almost totally re-sealed so as to inhibit evaporation of solution therefrom. It has been experimentally found that other shapes such as popularly used “X-shaped” or “H-shaped” cuts do not provide the degree of re-sealing as provided by closure 34 disclosed herein. It may be postulated that the stiffness of stopper 40 in the area surrounding slit 50 is important in allowing slit 50 to be so effectively re-closed, and in an exemplary embodiment, stopper 40 comprises a resealable elastomeric material selected from the group consisting of synthetic rubber, silicone rubber, thermoplastic elastomeric and the like. In particular, closure 34 comprises a puncturable, re-sealable material having a durometer value in a range selected to provide a sufficiently stiff layer ability to withstand multiple punctures, and in a range selected to provide sufficient flexibility to reseal and thereby inhibit evaporation. In an advantageous embodiment, vial 34 has dimensions 3-4 cm in height and between 1-2 cm in diameter, containing an amount of calibration or control solution in the range of about 1-2.5 mL. In such an instance, cap 38 is about 1-2 cm in diameter and stopper 40 is about 1-1.5 cm in diameter and 3-5 mm in height and is formed out of a thermoplastic elastomeric material. Slit 50 is about 4-6 mm in length for a probe of diameter about 1.5-2.5 mm in diameter. Metallic foil 46 may be made of a number of materials, however, an aluminum foil of about 3-5 mils thickness has been advantageously employed, foil 45 also being coated on its underside with a polyethylene sealant layer causing it to secure to circular band portion 44 of stopper 40 when processed, or when induction heated.

As explained previously, closure 34 is adapted for use in a manner that advantageously minimizes evaporation from a solution vial: (1) prior to aspiration of solution therefrom as a result of the non-air permeable foil 46; (2) subsequent to aspiration of solution therefrom as a result of the re-sealing nature of stopper 40; further, (3) foil 46 being recessed away from the top of cap 38 for protection from accidental tearing; and, (4) closure 34 being formed of relatively low-cost materials.

It should be readily understood by those persons skilled in the art that the present invention is susceptible of a broad utility and application. Many embodiments and adaptations of the present invention other than those herein described, as well as many variations, modifications and equivalent arrangements will be apparent from or reasonably suggested by the present invention and the foregoing description thereof, without departing from the substance or scope of the present invention.

Accordingly, while the present invention has been described herein in detail in relation to specific embodiments, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements, the present invention being limited only by the claims appended hereto and the equivalents thereof.

We claim:

1. A closure for sealing a vial, the closure comprising:
a cap element having an opening to permit a probe to pass therethrough; and,
a stopper element having a lowermost circular trunk portion with a closed bottom, the bottom having a line slit formed therethrough, the trunk joined to an outwardly extending upper shoulder portion, the shoulder portion depending from an outermost circular band portion, the circular band portion having an open top,
wherein the cap element is sized to fit over and retain the stopper element.

2. The closure of claim 1 wherein the closure further comprises a foil over the open top of the circular band.

3. The closure of claim 2 wherein the foil is metallic and is coated with a polyethylene sealant layer to seal it to the circular band.

4. The closure of claim 1 wherein the upper shoulder portion is sized to prevent the stopper from being fully inserted into the vial.

5. The closure of claim 1 wherein the stopper element is made of a puncturable, re-sealable material.

6. The closure of claim 5 wherein said puncturable, re-sealable material has its durometer in a range selected to provide a sufficiently stiff layer ability to withstand multiple punctures, and in a range selected to provide sufficient flexibility to reseal and thereby inhibit evaporation.

7. The closure of claim 1 wherein the cap is threaded.
